(19) United States
(12) Patent Application Publication
(10) Pub. No.: US 2011/0033545 A1
(43) Pub. Date: Feb. 10, 2011

(54) TOPICAL PHARMACEUTICAL PREPARATIONS HAVING BOTH A NANOPARTICLE SOLUTION AND A NANOPARTICLE SUSPENSION AND METHODS FOR THE TREATMENT OF ACUTE AND CHRONIC PAIN THEREWITH

(75) Inventor: Changjin Wang, San Diego, CA (US)

Correspondence Address: FOLEY & LARDNER LLP, 975 PAGE MILL ROAD, Palo Alto, CA, 94304, US

(73) Assignee: ABsize, Inc., Osaka (JP)

(21) Appl. No.: 12/844,369

(22) Filed: Jul. 27, 2010

Related U.S. Application Data
(60) Provisional application No. 61/273,473, filed on Aug. 6, 2009.

(51) Int. Cl. A61K 31/5415; A61K 9/14; A61K 31/192; A61K 31/197; A61P 29/00; A61P 23/02

(52) U.S. Cl. 424/489; 514/226.5; 514/570; 514/567

(57) ABSTRACT

This invention relates to topical pharmaceutical preparations and methods for the treatment of acute and chronic pain and inflammation therewith. The preparations have a saturated solution of an active pharmaceutical ingredient in a solvent therefor in intimate combination and contact with a suspension of nanoparticles of the active pharmaceutical ingredient in the solvent, and a pharmaceutically acceptable carrier therefor, and are administered topically.

### CROSS-REFERENCE TO RELATED APPLICATION

This application claims the benefit under 35 U.S.C. §119(e) of U.S. Provisional Patent Application Ser. No. 61/273,473 filed Aug. 6, 2009 which application is incorporated herein by reference in its entirety.

### FIELD OF THE PRESENT INVENTION

This invention relates to topical pharmaceutical preparations. This invention also relates to methods for the treatment of acute and chronic pain and inflammation therewith. The preparations of this invention have a saturated solution of an active pharmaceutical ingredient in a solvent therefor in intimate combination and contact with a suspension of nanoparticles of the active pharmaceutical ingredient in the solvent, and a pharmaceutically acceptable carrier therefor suitable for topical administration.

### BACKGROUND OF THE PRESENT INVENTION

The art is replete with the ways of preparing topical pharmaceutical preparations as well as with dealing with pain and inflammation, both in humans and animals. The medical literature and patents around the world describe many ways in which these conditions can be treated, with greater or lesser success in any given situation. More recently, many researchers have focused on using small particles of the active pharmaceutical ingredient in hope of achieving better clinical success with the enhanced administration that the smaller, so called nanoparticles, of the active pharmaceutical ingredient afford.

For example, US published application No. 2008/0237376 assigned to ABsize Inc. describes a technique for creating nanoparticles of an active pharmaceutical ingredient with a laser beam. Related published applications assigned to ABsize are US published applications 20070284769 and 20080217445. Other laser beam nanosizing techniques are shown by Kawakami et al. in US published applications 20060257489, 20070114306 and 20070152360. However, while these publications describe various techniques for creating nanoparticles, and using them, for example, in preparing injectable agents, they do not describe topical preparations or how topical preparations can be effectively utilized in the treatment of acute pain and inflammation.

It is, therefore, an object of the present invention to provide pharmaceutical preparations that are topically applied as well as to provide methods for the treatment of acute and chronic pain and inflammation therewith that have higher concentrations of the active pharmaceutical ingredient. It is contemplated that the pharmaceutical preparations of this invention will allow lower dosages of the active agent to be used in comparison with the doses normally administered systemically to achieve efficacious results. It is further contemplated that the use of lower dosages of the active agent will result in better clinical efficacy and reduced side effects, which allow for longer-term treatment than current therapies.

### SUMMARY OF THE INVENTION

This invention relates to pharmaceutical preparations as well as to methods for the treatment of acute and chronic pain and inflammation, and their related symptoms.

In one embodiment, this invention relates to pharmaceutical preparations comprising a therapeutically effective amount of a nanosized non-steroidal anti-inflammatory drug, and a pharmaceutically acceptable carrier therefor suitable for topical administration. The preparations are administered topically for the treatment of acute and chronic pain and inflammation, and their related symptoms.

In another embodiment, the pharmaceutical preparations of this invention further comprise an effective amount of a local anesthetic.

The non-steroidal anti-inflammatory drug is nanosized to nanoparticles of less than 1000 nm, predominately in the range of 200-500 nm or 1-200 nm, preferably 10-100 nm or 50-100 nm, and most preferably having a mean particle size of about 40-60 nm (as measured along it longest axis). It should be understood that the non-steroidal anti-inflammatory drug is itself nanosized, as opposed to where the medicinal ingredient is absorbed onto an inert nanosized carrier.

The preparations of this invention are useful in the treatment of acute and/or chronic pain as a result of inflammation associated with, for example, rheumatoid arthritis, osteoarthritis, inflammatory arthropathies, gout and pseudogout, dysmenorrhea, metastatic bone pain, headache and migraine, postoperative pain, post-herpetic neuralgia, neuropathic pains, soft-tissue injuries, strains, sprains, contusions, tendonitis or bursitis of the shoulder, elbow, wrist or knee, Carpal tunnel syndrome, lateral epicondylosis, lower back pains and injury, and the like. It is understood that this invention is not directed to novel non-steroidal anti-inflammatory agents. Rather, this invention is directed to use of known agents that are being nanosized so as to improve their efficacy for topical administration. It is also understood that this invention is not treating the diseases referenced above but, rather, are treating the inflammation and pain associated therewith.

### DETAILED DESCRIPTION OF SPECIFIC EMBODIMENTS OF THE PRESENT INVENTION

In one embodiment of this invention, the pharmaceutical preparations comprise a therapeutically effective amount of nanosized particles of a solid medicament and, in particular, a non-steroidal anti-inflammatory drug selected from the group consisting of aceclofenac, alminoprofen, apazone, aspirin, benoxaprofen, butibufen, carprofen, dexketoprofen, diclofenac, difenpiramide, diflunisal, droxicam, enbufen, etodolac, fenoprofen, flufenamic acid, flurbiprofen, ibuprofen, indomethacin, indoprofen, ketoprofen, ketorolac, lornoxicam, meclofenamic acid, mefenamic acid, meloxicam, nabumetone, naproxen, oxaprozin, phenylbutazone, piroxicam, pirprofen, pranoprofen, salicylic acid, sulindac, suprofen, tenoxicam, tiaprofenic acid, and tolmetin, and the pharmaceutically acceptable salts, and esters thereof, and a pharmaceutically acceptable carrier therefor suitable for topical administration. The preferred class of the non-steroidal anti-inflammatory drugs is the oxicam class of NSAIDs. The presently preferred non-steroidal anti-inflammatory drugs are diclofenac, ketoprofen, ketorolac, and piroxicam, with the presently preferred non-steroidal anti-inflammatory drug being piroxicam.

In another embodiment of the present invention, the pharmaceutical preparations have a therapeutically effective amount of nanosized particles of a non-steroidal anti-inflammatory drug as set forth above in combination with a local anesthetic selected from the group consisting of articaine, benzocaine, bupivacaine, dibucaine, etidocaine, levobupivacaine, lidocaine, mepivacaine, piperocaine, prilocaine, ropivacaine, tetracaine, and trimecaine. The presently preferred local anesthetics are bupivacaine, lidocaine, prilocaine, and tetracaine, with the presently preferred local anesthetic being lidocaine.

The nanoparticles of the non-steroidal anti-inflammatory drug can be prepared by any suitable nanosizing technique, preferably through use of a laser beam, more preferably a pulse laser beam having an excitation intensity of 1 to 1,000 mJ/cm². Laser beam nanosizing techniques are shown by US published application No. 20080237376 assigned to ABsize, Inc. and related US published applications 20070284769 and 20080217445, also assigned to ABsize, Inc. Other laser beam nanosizing techniques are shown by Kawakami et al. in US published applications 20060257489, 20070114306 and 20070152360. All of these publications are incorporated herein by reference in their entirety.

The preferred nanosizing technique is the laser beam technique of US published application 20080237376, That technique is referred to as the LiNTEC technology (Laser-induced Nanolization Technology). Using that technique or technology in the practice of this invention would require determining, according to methods well known in the art, the absorption wavelengths of the particular non-steroidal anti-inflammatory drug being used and irradiating that drug with wavelengths that are absorbed by the drug, under the conditions shown by US published application 20080237376. Generally that would mean irradiating the non-steroidal anti-inflammatory drug in an appropriate solvent, such as water, with a laser beam having a wavelength of 200-800 nm, preferably a pulse laser having a pulse width of ranging from about several femtoseconds to about several hundred nanoseconds. The absorption causes the non-steroidal anti-inflammatory drug to become nanosized such that a portion of the theretofore non-soluble non-steroidal anti-inflammatory drug goes into solution, compared to the amount in solution before the irradiation took place, while leaving a portion of the insoluble nanosized particles of the non-steroidal anti-inflammatory drug suspended in the solvent. The solution and the suspension are in intimate contact and combination with one another, and it is that combination that is used directly without filtration in the preparation of the topical preparations of the present invention. The LiNTEC technology is useful for generating nanoparticles having a mean particle size of 40-60 nm, which are particularly suited for use in the practice of the present invention.

The pharmaceutical preparations of this invention are intended to be administered topically by any means known in the art. That includes, for example, creams, gels, lotions, ointments and transdermal patches. It is essential that the pharmaceutical preparations of the present invention be administered locally so as to minimize, to the extent possible, unwanted side effects that might arise from systemic administration.

With regard to the non-steroidal anti-inflammatory drugs listed above, this invention contemplates using about 20 mg to about 2,000 mg/day of aceclofenac, preferably about 200 mg to about 1,000 mg/day, with a specific dose of about 600 mg/day; about 9 mg to about 900 mg/day of alminoprofen, preferably about 90 mg to about 450 mg/day, with a specific dose of about 270 mg/day; about 30 mg to about 3,000 mg/day of apazone, preferably about 300 mg to about 1,500 mg/day, with a specific dose of about 900 mg/day; about 400 mg to about 40,000 mg/day of aspirin, preferably about 4,000 mg to about 20,000 mg/day, with a specific dose of about 12,000 mg/day; about 60 mg to about 6,000 mg/day of benoxaprofen, preferably about 600 mg to about 3,000 mg/day, with a specific dose of about 1,800 mg/day; about 300 mg to about 30,000 mg/day of butibufen, preferably about 3,000 mg to about 15,000 mg/day, with a specific dose of about 9,000 mg/day; about 10 mg to about 1,000 mg/day of carproen, preferably about 100 mg to about 500 mg/day, with a specific dose of about 300 mg/day; about 5 mg to about 500 mg/day of dexketoprofen, preferably about 50 mg to about 250 mg/day, with a specific dose of about 150 mg/day; about 20 mg to about 2,000 mg/day of diclofenac, preferably about 200 mg to about 1,000 mg/day, with a specific dose of about 600 mg/day; about 20 mg to about 2,000 mg/day of difenpiramide, preferably about 200 mg to about 1,000 mg/day, with a specific dose of about 600 mg/day; about 100 mg to about 10,000 mg/day of diflunisal, preferably about 1,000 mg to about 5,000 mg/day, with a specific dose of about 3,000 mg/day; about 2 mg to about 200 mg/day of droxicam, preferably about 20 mg to about 100 mg/day, with a specific dose of about 60 mg/day; about 100 mg to about 10,000 mg/day of etodolac, preferably about 1,000 mg to about 5,000 mg/day, with a specific dose of about 3,000 mg/day; about 30 mg to about 3,000 mg/day of fenbufen, preferably about 300 mg to about 1,500 mg/day, with a specific dose of about 900 mg/day; about 120 mg to about 12,000 mg/day of fenoprofen, preferably about 1,200 mg to about 6,000 mg/day, with a specific dose of about 3,600 mg/day; about 60 mg to about 6,000 mg/day of flufenamic acid, preferably about 600 mg to about 3,000 mg/day, with a specific dose of about 1,800 mg/day; about 30 mg to about 3,000 mg/day of flurbiprofen, preferably about 300 mg to about 1,500 mg/day, with a specific dose of about 900 mg/day; about 300 mg to about 30,000 mg/day of ibuprofen, preferably about 3,000 mg to about 15,000 mg/day, with a specific dose of about 9,000 mg/day; about 10 mg to about 1,000 mg/day of indomethacin, preferably about 100 mg to about 500 mg/day, with a specific dose of about 300 mg/day; about 80 mg to about 8,000 mg/day of indoprofen, preferably about 800 mg to about 4,000 mg/day, with a specific dose of about 2,400 mg/day; about 30 mg to about 3,000 mg/day of ketoprofen, preferably about 300 mg to about 1,500 mg/day, with a specific dose of about 900 mg/day; about 4 mg to about 400 mg/day of ketorolac, preferably about 40 mg to about 200 mg/day, with a specific dose of about 120 mg/day; about 1.6 mg to about 160 mg/day of lornoxicam, preferably about 16 mg to about 80 mg/day, with a specific dose of about 48 mg/day; about 30 mg to about 3,000 mg/day of meclofenamic acid, preferably about 300 mg to about 1,500 mg/day, with a specific dose of about 900 mg/day; about 100 mg to about 10,000 mg/day of mefenamic acid, preferably about 1,000 mg to about 5,000 mg/day, with a specific dose of about 3,000 mg/day; about 1.5 mg to about 150 mg/day of meloxicam, preferably about 15 mg to about 75 mg/day, with a specific dose of about 45 mg/day; about 100 mg to about 10,000 mg/day of nabumetone, preferably about 1,000 mg to about 5,000 mg/day, with a specific dose of about 3,000 mg/day; about 150 mg to about 15,000 mg/day of naproxen, preferably about 1,500 mg to about 7,500 mg/day, with a specific dose of about 4,500 mg/day; about 120 mg to about 12,000 mg/day of oxaprozin, preferably about 1,200 mg to about 6,000 mg/day, with a specific dose of about 3,600 mg/day; about 30 mg to about 3,000 mg/day of phenylbutazone, preferably about 300 mg to about 1,500 mg/day, with a specific dose of about 900 mg/day; about 2 mg to about 200 mg/day of piroxicam, preferably about 20 mg to about 100 mg/day, with a specific dose of about 60 mg/day; about 60 mg to about 6,000 mg/day of pirprofen, preferably about 600 mg to about 3,000 mg/day, with a specific dose of about 1,800 mg/day; about 400 mg to about 40,000 mg/day of salicylic acid, preferably about 4,000 mg to about 20,000 mg/day, with a specific dose of about 12,000 mg/day; about 40 mg to about 4,000 mg/day of sulindac, preferably about 400 mg to about 2,000 mg/day, with a specific dose of about 1,200 mg/day; about 2 mg to about 200 mg/day of tenoxicam, preferably about 20 mg to about 100 mg/day, with a specific dose of about 60 mg/day; about 60 mg to about 6,000 mg/day of tiaprofenic acid, preferably about 600 mg to about 3,000 mg/day, with a specific dose of about 1,800 mg/day; and about 180 mg to about 18,000 mg/day of tolmetin, preferably about 1,800 mg to about 9,000 mg/day, with a specific dose of about 5,400 mg/day. The pharmaceutically acceptable salts, and acids, as are known in the art, may be used, the daily dose being such as to provide the amounts of the drugs set forth above. Although the topical formulations of Examples 10-21 below are given in percentages of the non-steroidal anti-inflammatory drug and the local anesthetic in the formulations, the formulations are nonetheless intended to deliver the dosage amounts of the non-steroidal anti-inflammatory drug as set forth above.

With regard to the local anesthetics listed above, this invention contemplates using about 20 mg to about 2,000 mg/day of articaine, preferably about 200 mg to about 1,000 mg/day, with a specific dose of about 600 mg/day; about 17.5 mg to about 1,750 mg/day of bupivacaine, preferably about 175 mg to about 875 mg/day, with a specific dose of about 525 mg/day; about 30 mg to about 3,000 mg/day of etidocaine, preferably about 300 mg to about 1,500 mg/day, with a specific dose of about 900 mg/day; about 30 mg to about 3,000 mg/day of levobupivacaine, preferably about 300 mg to about 1,500 mg/day, with a specific dose of about 900 mg/day; about 30 mg to about 3,000 mg/day of lidocaine, preferably about 300 mg to about 1,500 mg/day, with a specific dose of about 900 mg/day; about 40 mg to about 4,000 mg/day of mepivacaine, preferably about 400 mg to about 2,000 mg/day, with a specific dose of about 1,200 mg/day; about 8 mg to about 800 mg/day of prilocaine, preferably about 80 mg to about 400 mg/day, with a specific dose of about 240 mg/day; about 20 mg to about 2,000 mg/day of ropivacaine, preferably about 200 mg to about 1,000 mg/day, with a specific dose of about 600 mg/day; and about 1 mg to about 100 mg/day of tetracaine, preferably about 10 mg to about 50 mg/day, with a specific dose of about 30 mg/day. Although the topical formulations of Examples 10-21 below are given in percentages of the non-steroidal anti-inflammatory drug and the local anesthetic in the formulations, the formulations are nonetheless intended to deliver the dosage amounts of the local anesthetic as set forth above.

In the above dosing ranges, it is understood that the amount of ingredient recited is the amount loaded to effect topical administration to the patient. The amount of ingredient actually delivered to the patient will be less than the amount loaded and will depend upon factors such as the rate of drug penetration through the skin, the total area of application, the duration of application and other factors all of which are well known in the art. Although broad ranges for each ingredient have been given above, it is anticipated that, by using nanoparticles of the non-steroidal anti-inflammatory drug, more drug can be delivered through the skin than that by using normal particle sizes. It is well known that only a small fraction (typically 1-5%) of a loaded pharmaceutical dose in a topical preparation or a transdermal patch is able to penetrate through the skin, and the process is driven by concentration gradients. By using nanoparticles of a non-steroidal anti-inflammatory drug, a super saturated concentration and suspension can be achieved, which may be used directly to prepare suitable pharmaceutical formulations for use in topical preparations or transdermal patches. This type of nanoparticle-drug topical preparations and transdermal patches are anticipated to have enhanced skin penetration and permeability in comparison with normal topical preparations and transdermal patches, resulting in enhanced drug delivery capability, more flexible and suitable dose regimens (e.g. smaller patch size), and more importantly clinical benefits. It is contemplated that such nanoparticle pharmaceutical preparation for topical and transdermal delivery will provide better clinical efficacy than that of normal particle preparations for topical and trandermal delivery, while retaining limited systemic exposure and reduced side effect profile in comparison with systemic delivery routes, such as oral dosage forms or injections.

The preparations of this invention may be used daily, weekly or even monthly, depending in part upon the need of each particular individual and the topical dosage form of the drug utilized. Preferably, the preparations of this invention can be applied daily, once a week, once every two weeks, or once a month. Once the beneficial results have been verified, the individual may be placed on a maintenance regimen where the therapy will be administered periodically as needed to maintain those beneficial results without necessarily having the same administration regimen as when the therapy was initially undertaken. This, as would be expected, may vary from individual to individual.

All of the specifically named non-steroidal anti-inflammatory drugs and local anesthetics of this invention are well known. All or some are commercially available, generally from multiple sources, and, if not, methods for the chemical manufacture thereof have been described in the patent literature and thus need not be described further herein. These ingredients may be used in any chemical form known in the art to be suitable for use in pharmaceutical preparations, for example, the acid form (e.g., naproxen) or a pharmaceutically acceptable salt form (e.g., naproxen sodium).

The preparations of the this invention include any pharmaceutically acceptable carrier suitable for topical delivery as would be known to one skilled in this art. The methods for the pharmaceutical manufacture of the preparations of the present invention into the various topical dosage forms suitable for use in this invention are well known to those skilled in this art (as shown, for example, in Remington's Pharmaceutical Sciences, 18^{th} Edition, 1990) and thus need not be described further herein. Each of the topical delivery forms will deliver a therapeutic amount of the drug or mixture of drugs found in the preparations of this invention to the site or sites where they are intended to be absorbed locally over some given period of time to achieve the beneficial effects of this invention. The delivery forms may be prepared for prompt or immediate release of the ingredients of the preparations of the present invention or for controlled or sustained release over a longer period of time.

Any type of transdermal patch can be used, such as matrix patches where the NSAID is in the adhesive layer or a reservoir patch, etc. The patch also contains typical inactive ingredients for a pharmaceutical topical formulation including, e.g., dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacrylate, D-sorbitol, tartaric acid, and urea.

### EXAMPLES

The preparations of this invention, how they are prepared, including nanosizing of the non-steroidal anti-inflammatory drug, and various topical delivery forms of the present invention are exemplified by the following specific embodiments.

### Example 1

LiNTEC NANOSIZING TECHNIQUE: solid particles of active pharmaceutical ingredient(s), such as an NSAID and/or a local anesthetic as described above, is/are dispersed into an aqueous solvent such as water, which is vortexed and/or sonicated with some particles going into solution and some particles remaining as solid. This solution and suspension of the active pharmaceutical ingredient(s) are then subjected to the LiNTEC nanosizing technique utilizing laser irradiation with appropriate wavelength irradiation based upon the particular pharmaceutical agents(s) being used. The resulting preparation is a solution and nanoparticle suspension of the active pharmaceutical ingredient(s) with nanoparticle size ranging from 5 to 500 nm, preferably from 10 to 200 nm, and most preferably from 20-100 nm. This preparation is then used for pharmaceutical formulations appropriate for topical and/or transdermal delivery.

### Example 2

### Patch with Piroxicam Only

A transdermal patch for treating acute and chronic pain and inflammation in the human is prepared having an adhesive material containing 60 mg of piroxicam nanoparticle suspension/solution prepared according to the procedure described in Example 1 in an aqueous base. It is intended to be applied to the skin in the area where the pain is felt most like the knee, shoulder, low back, by the patient, once every day, every two days, every three days, or every 5-7 days. Instead of having 60 mg of piroxicam, the patch may have 30 mg, 40 mg, 50 mg, 70 mg, 80 mg or 100 mg of piroxicam. The size of the patch can be 2 cm×3 cm, 3 cm×4cm, 5 cm×7 cm, 7 cm×10 cm, 10 cm×14 cm, etc.

### Example 3

### Patch with Piroxicam and Lidocaine

A transdermal patch for treating acute and chronic pain and inflammation in the human is prepared having an adhesive material containing 60 mg of piroxicam nanoparticle suspension/solution nanoparticle suspension/solution prepared according to the technique described in Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 60 mg of piroxicam and 900 mg of lidocaine, the patch may have 30 mg, 40 mg, 50 mg, 70 mg, 80 mg or 100 mg of piroxicam and 200 mg, 300 mg, 400 mg, 500 mg, 600 mg, 700 mg, 800 mg, 1,000 mg, 1,500 mg or 2,000 mg of lidocaine in any medically appropriate combination. The size of the patch can be 3 cm×5 cm, 5 cm×7 cm, 7 cm×10 cm, 10 cm×14 cm, etc.

### Example

4

### Patch with Meloxicam Only

A transdermal patch for treating acute and chronic pain and inflammation in the human is prepared having an adhesive material containing 45 mg of meloxicam nanoparticle suspension/solution prepared according to the technique described in Example 1 in an aqueous base. It is intended to be applied to the skin in the area where the pain is felt most like the knee, shoulder, low back, by the patient, once every day, every two days, every three days, or every 5-7 days. Instead of having 45 mg of meloxicam, the patch may have 5 mg, 15 mg, 30 mg, 55 mg, 70 mg, 85 mg, 100 mg, or 150 mg of meloxicam. The size of the patch can be 3×5 cm, 5 cm×7 cm, 7 cm×10 cm, 10 cm×14 cm, etc.

### Example 5

### Patch with Meloxicam and Lidocaine

A transdermal patch for treating acute and chronic pain and inflammation in the human is prepared having an adhesive material containing 45 mg of meloxicam nanoparticle suspension/solution prepared according to the technique described in Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 45 mg of meloxicam and 900 mg of lidocaine, the patch may have 5 mg, 15 mg, 30 mg, 55 mg, 70 mg, 85 mg, 100 mg, or 150 mg of meloxicam and 200 mg, 300 mg, 400 mg, 500 mg, 600 mg, 700 mg, 800 mg, 1,000 mg, 1,500 mg or 2,000 mg of lidocaine in any medically appropriate combination. The size of the patch can be 5 cm×7 cm, 7 cm×10 cm, 10 cm×14 cm, etc.

### Example 6

### Patch with Ketoprofen Only

A transdermal patch for treating acute and chronic pain and inflammation in the human is prepared having an adhesive material containing 900 mg of ketoprofen nanoparticle suspension/solution prepared according to the technique described in Example 1 in an aqueous base. It is intended to be applied to the skin in the area where the pain is felt most like the knee, shoulder, low back, by the patient, once every day, every two days, every three days, or every 5-7 days. Instead of having 900 mg of ketoprofen, the patch may have 100 mg, 200 mg, 300 mg, 400 mg, 500 mg, 600 mg, 700 mg, 800 mg, 900 mg, 1,000 mg or 1,500 mg of ketoprofen. The size of the patch can be 5 cm×7 cm, 7 cm×10 cm, 10 cm×14 cm, etc.

### Example 7

### Patch with Ketoprofen and Lidocaine

A transdermal patch for treating acute and chronic pain and inflammation in the human is prepared having an adhesive material containing 900 mg of ketoprofen nanoparticle suspension/solution prepared according to the technique of Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 900 mg of ketoprofen and 900 mg of lidocaine, the patch may have 100 mg, 200 mg, 300 mg, 400 mg, 500 mg, 600 mg, 700 mg, 800 mg, 900 mg, 1,000 mg or 1,500 mg of ketoprofen and 200 mg, 300 mg, 400 mg, 500 mg, 600 mg, 700 mg, 800 mg, 1,000 mg, 1,500 mg or 2,000 mg of lidocaine in any medically appropriate combination. The size of the patch can be 5 cm×7 cm, 7 cm×10 cm, 10 cm×14 cm, etc.

### Example 8

### Patch with Diclofenac Only

A transdermal patch for treating acute and chronic pain and inflammation in the human is prepared having an adhesive material containing 600 mg of diclofenac nanoparticle suspension/solution prepared according to the technique described in Example 1 in an aqueous base. It is intended to be applied to the skin in the area where the pain is felt most like the knee, shoulder, low back, by the patient, once every day, every two days, every three days, or every 5-7 days. Instead of having 600 mg of diclofenac, the patch may have 100 mg, 200 mg, 300 mg, 400 mg, 500 mg, 700 mg, 800 mg, 900 mg, 1,000 mg or 1,500 mg of diclofenac. The size of the patch can be 5 cm×7 cm, 7cm×10 cm, 10 cm×14 cm, etc.

### EXAMPLE 9

### Patch with Diclofenac and Lidocaine

A transdermal patch for treating acute and chronic pain and inflammation in the human is prepared having an adhesive material containing 600 mg of diclofenac nanoparticle suspension/solution prepared according to the technique described in Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 600 mg of diclofenac and 900 mg of lidocaine, the patch may have 100 mg, 200 mg, 300 mg, 400 mg, 500 mg, 700 mg, 800 mg, 900 mg, 1,000 mg or 1,500 mg of diclofenac and 200 mg, 300 mg, 400 mg, 500 mg, 600 mg, 700 mg, 800 mg, 1,000 mg, 1,500 mg or 2,000 mg of lidocaine in any medically appropriate combination. The size of the patch can be 5 cm×7 cm, 7 cm×10 cm, 10 cm×14 cm, etc.

### Example 10

### Gel with Piroxicam Only

A topical gel preparation for treating acute and chronic pain and inflammation in the human is prepared having a formulation containing 0.5% piroxicam nanoparticle suspension/solution prepared in accordance with the technique of Example 1 in an aqueous base. The gel is intended to be applied to the skin in the area where the pain is felt most like the knee, shoulder, low back, by the patient, once a day, twice a day, or 3-5 times a day. Instead of having 0.5% piroxicam, the gel may have 0.1%, 0.25%, 0.75%, 1%, 2%, 3%, 4%, 5% or 10% of piroxicam.

### Example 11

### Gel with Piroxicam and Lidocaine

A topical gel preparation for treating acute and chronic pain and inflammation in the human is prepared having a formulation containing 0.5% piroxicam nanoparticle suspension/solution prepared according to the technique described in Example 1 and 5% lidocaine in an aqueous base. The gel is intended to be applied to the skin in the area where the pain is felt most like the knee, shoulder, low back, by the patient, once a day, twice a day, or 3-5 times a day. Instead of having 0.5% piroxicam and 5% lidocaine, the gel may have 0.1%, 0.25%, 0.75%, 1%, 2%, 3%, 4%, 5% or 10% of piroxicam and 1%, 2%, 3%, 4%, 5%, 6%, 8% or 10% of lidocaine in any medically appropriate combination.

### Example 12

### Gel with Meloxicam Only

A topical gel preparation for treating acute and chronic pain and inflammation in the human is prepared having a formulation containing 0.5% meloxicam nanoparticle suspension/solution prepared according to the technique described in Example 1 in an aqueous base. The gel is intended to be applied to the skin in the area where the pain is felt most like the knee, shoulder, low back, by the patient, once a day, twice a day, or 3-5 times a day. Instead of having 0.5% meloxicam, the gel may have 0.1%, 0.25%, 0.75%, 1%, 2%, 3%, 4%, 5% or 10% meloxicam.

### Example 13

### Gel with Meloxicam and Lidocaine

A topical gel preparation for treating acute and chronic pain and inflammation in the human is prepared having a formulation containing 0.5% meloxicam nanoparticle suspension/solution prepared according to the technique described in Example 1 and 5% lidocaine in an aqueous base. The gel is intended to be applied to the skin in the area where the pain is felt most like the knee, shoulder, low back, by the patient, once a day, twice a day, or 3-5 times a day. Instead of having 0.5% meloxicam and 5% lidocaine, the gel may have 0.1%, 0.25%, 0.75%, 1%, 2%, 3%, 4%, 5% or 10% of meloxicam and 1%, 2%, 3%, 4%, 5%, 6%, 8% or 10% lidocaine in any medically appropriate combination.

### Example 14

### Gel with Ketoprofen Only

A topical gel preparation for treating acute and chronic pain and inflammation in the human is prepared having a formulation containing 5% ketoprofen nanoparticle suspension/solution prepared according to the technique described in Example 1 in an aqueous base. The gel is intended to be applied to the skin in the area where the pain is felt most like the knee, shoulder, low back, by the patient, once a day, twice a day, or 3-5 times a day. Instead of having 5% ketoprofen, the gel may have 1%, 2%, 3%, 4% 6%, 7%, 8%, 9% or 10% of ketoprofen.

EXAMPLE 15

### Gel with Ketoprofen and Lidocaine

A topical gel preparation for treating acute and chronic pain and inflammation in the human is prepared having a formulation containing 5% ketoprofen nanoparticle suspension/solution prepared according to the technique described in Example 1 and 5% lidocaine in an aqueous base. The gel is intended to be applied to the skin in the area where the pain is felt most like the knee, shoulder, low back, by the patient, once a day, twice a day, or 3-5 times a day. Instead of having 5% ketoprofen and 5% lidocaine, the gel may have 1%, 2%, 3%, 4%, 6%, 7%, 8%, 9% or 10% of ketoprofen and 1%, 2%, 3%, 4%, 6%, 8% or 10% of lidocaine in any medically appropriate combination.

### Example 16

### Cream with Piroxicam Only

A topical cream preparation for treating acute and chronic pain and inflammation in the human is prepared having a formulation containing 0.5% piroxicam nanoparticle suspension/solution prepared according to the technique described in Example 1 in an aqueous base. The cream is intended to be applied to the skin in the area where the pain is felt most like the knee, shoulder, low back, by the patient, once a day, twice a day, or 3-5 times a day. Instead of having 0.5% piroxicam, the cream may have 0.1%, 0.2%, 0.3%, 0.4%, 0.6%, 0.7%, 0.8%, 0.9%, 1%, 2%, 3%, 4% or 5% of piroxicam.

### Example 17

### Cream with Piroxicam and Lidocaine

A topical cream preparation for treating acute and chronic pain and inflammation in the human is prepared having a formulation containing 0.5% piroxicam nanoparticle suspension/solution prepared according to the technique of Example 1 and 5% lidocaine in an aqueous base. It is intended to be applied to the skin in the area where the pain is felt most like the knee, shoulder, low back, by the patient, once a day, twice a day, or 3-5 times a day. Instead of having 0.5% piroxicam and 5% lidocaine, the cream may have 0.1%, 0.2%, 0.3%, 0.4%, 0.6%, 0.7%, 0.8%, 0.9%, 1%, 2%, 3%, 4% or 5% piroxiacm and 1%, 2%, 3%, 4%, 6%, 8% or 10% of lidocaine in any medically appropriate combination.

### Example 18

### Cream with Meloxicam Only

A topical cream preparation for treating acute and chronic pain and inflammation in the human is prepared having a formulation containing 0.5% meloxicam nanoparticle suspension/solution prepared according to the technique of Example 1 in an aqueous base. The cream is intended to be applied to the skin in the area where the pain is felt most like the knee, shoulder, low back, by the patient, once a day, twice a day, or 3-5 times a day. Instead of having 0.5% meloxicam, the cream may have 0.1%, 0.2%, 0.3%, 0.4%, 0.6%, 0.7%, 0.8%, 0.9%, 1%, 2%, 3%, 4%, or 5% of meloxicam.

### Example 19

### Cream with Meloxicam and Lidocaine

A topical cream preparation for treating acute and chronic pain and inflammation in the human is prepared having a formulation containing 0.5% meloxicam nanoparticle suspension/solution prepared according to the technique of Example 1 and 5% lidocaine in an aqueous base. The cream is intended to be applied to the skin in the area where the pain is felt most like the knee, shoulder, low back, by the patient, once a day, twice a day, or 3-5 times a day. Instead of having 0.5% meloxicam and 5% lidocaine, the cream may have 0.1%, 0.2%, 0.3%, 0.4%, 0.6%, 0.7%, 0.8%, 0.9%, 1%, 2%, 3%, 4%, or 5% of meloxicam and 1%, 2%, 3%, 4%, 6%, 8% or 10% of lidocaine in any medically appropriate combination.

### Example 20

### Cream with Ketoprofen Only

A topical cream preparation for treating acute and chronic pain and inflammation in the human is prepared having a formulation containing 5% ketoprofen nanoparticle suspension/solution prepared according to the technique of Example 1 in an aqueous base. The cream is intended to be applied to the skin in the area where the pain is felt most like the knee, shoulder, low back, by the patient, once a day, twice a day, or 3-5 times a day. Instead of having 5% ketoprofen, the cream may have 1%, 2%, 3%, 4%, 6%, 7%, 8%, 9% or 10% of ketoprofen.

### Example 21

### Cream with Ketoprofen and Lidocaine

A topical cream preparation for treating acute and chronic pain and inflammation in the human is prepared having a formulation containing 5% ketoprofen nanoparticle suspension/solution prepared according to the technique of Example 1 and 5% lidocaine in an aqueous base. The cream is intended to be applied to the skin in the area where the pain is felt most like the knee, shoulder, low back, by the patient, once a day, twice a day, or 3-5 times a day. Instead of having 5% ketoprofen and 5% lidocaine, the cream may have 1%, 2%, 3%, 4%, 6%, 7%, 8%, 9%, or 10% of ketoprofen and 1%, 2%, 3%, 4%, 6%, 8% or 10% of lidocaine in any medically appropriate combination.

### Example 22

### Reservoir Type Patch with Piroxicam Only

A transdermal patch for treating acute and chronic pain and inflammation in the human is prepared having an adhesive layer and porous membrane separated from a drug reservoir containing 100 mg of piroxicam nanoparticle suspension/solution prepared according to the technique of Example 1 in an aqueous base. The patch is intended to be applied to the skin in the area where the pain is felt most like the knee, shoulder, low back, by the patient, once every day, every two days, every three days, or every 5-7 days. Instead of having 100 mg piroxicam, the patch may have 30 mg, 40 mg, 50 mg, 60 mg, 80 mg or 150 mg, or 200 mg of piroxicam. The size of the patch can be 5 cm×7 cm, 7 cm×10 cm, 10 cm×14 cm, etc.

### Example 23

### Reservoir Type of Patch with Piroxicam and Lidocaine

A transdermal patch for treating acute and chronic pain and inflammation in the human is prepared having an adhesive layer and porous membrane separated from a drug reservoir containing 100 mg of piroxicam nanoparticle suspension/solution prepared according to the technique of Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 100 mg of piroxicam and 900 mg of lidocaine, the patch may have 20 mg, 30 mg, 40 mg, 50 mg, 60 mg, 80 mg, 150 mg, or 200 mg of piroxicam and 300 mg, 500 mg, 1,000 mg, 1,500 mg, 2,000 mg or 3,000 mg of lidocaine in any medically appropriate combination.

### Example 24

### Reservoir Type Patch with Meloxicam Only

A transdermal patch for treating acute and chronic pain and inflammation in the human is prepared which is comprised of an adhesive layer and porous membrane separated from a drug reservoir containing 75 mg of meloxicam nanoparticle suspension/solution in an aqueous base. The patch is intended to be applied to the skin in the area where the pain is felt most like the knee, shoulder, low back, by the patient, once every day, every two days, every three days, or every 5-7 days. Instead of having 75 mg of meloxicam, the patch may have 5 mg, 10 mg, 15 mg, 30 mg, 40 mg, 50 mg, 60 mg, 80 mg, 100 mg or 150 mg of meloxicam. The size of the patch can be 5 cm×7 cm, 7 cm×10 cm, 10 cm×14 cm, etc.

### Example 25

### Reservoir Type Patch with Meloxicam and Lidocaine

A transdermal patch for treating acute and chronic pain and inflammation in the human is prepared having an adhesive layer and porous membrane separated from a drug reservoir containing 75 mg of meloxicam nanoparticle suspension/solution prepared according to the technique of Example 1 and 900 mg of lidocaine in an aqueous base. The patch is intended to be applied to the skin in the area where the pain is felt most like the knee, shoulder, low back, by the patient, once every day, every two days, every three days, or every 5-7 days. Instead of having 75 mg of meloxicam and 900 mg of lidocaine, the patch may have 5 mg, 10 mg, 15 mg, 30 mg, 40 mg, 50 mg, 60 mg, 80 mg, 100 mg or 150 mg of meloxicam and 300 mg, 500 mg, 1,000 mg, 1,500 mg, 2,000 mg or 3,000 mg of lidocaine in any medically appropriate combination. The size of the patch can be 5 cm×7 cm, 7 cm×10 cm, 10 cm×14 cm, etc.

### Example 26

### Patch with Ketorolac Only

A transdermal patch for treating acute and chronic pain and inflammation in the human is prepared having an adhesive material containing 120 mg of ketorolac nanoparticle suspension/solution prepared according to the procedure described in Example 1 in an aqueous base. It is intended to be applied to the skin in the area where the pain is felt most like the knee, shoulder, low back, by the patient, once every day, every two days, every three days, or every 5-7 days. Instead of having 120 mg ketorolac the patch may have 40 mg, 60 mg, 80 mg, 100 mg, 140 mg, 160 mg, 180 mg or 200 mg of ketorolac. The size of the patch can be 2 cm×3 cm, 3 cm×4 cm, 5 cm×7 cm, 7 cm×10 cm, 10 cm×14 cm, etc.

### Example 27

### Patch with Ketorolac and Lidocaine

A transdermal patch for treating acute and chronic pain and inflammation in the human is prepared having an adhesive material containing 120 mg of ketorolac nanoparticle suspension/solution nanoparticle suspension/solution prepared according to the technique described in Example 1 and 900 mg of lidocaine in an aqueous base. It is intended to be applied to the skin in the area where the pain is felt most like the knee, shoulder, low back, by the patient, once every day, every two days, every three days, or every 5-7 days. Instead of having 120 mg of ketorolac and 900 mg of lidocaine, the patch may have 40 mg, 60 mg, 80 mg, 100 mg, 140 mg, 160 mg, 180 mg or 200 mg of ketorolac and 200 mg, 300 mg, 400 mg, 500 mg, 600 mg, 700 mg, 800 mg, 1,000 mg, 1,500 mg or 2,000 mg of lidocaine in any medically appropriate combination. The size of the patch can be 3 cm×5 cm, 5 cm×7 cm, 7 cm×10 cm, 10 cm×14 cm, etc.

### Example 28

### Patch with Piroxicam and Prilocaine

A transdermal patch for treating acute and chronic pain and inflammation in the human is prepared having an adhesive material containing 60 mg of piroxicam nanoparticle suspension/solution nanoparticle suspension/solution prepared according to the technique described in Example 1 and 240 mg of prilocaine in an aqueous base. It is intended to be applied to the skin in the area where the pain is felt most like the knee, shoulder, low back, by the patient, once every day, every two days, every three days, or every 5-7 days. Instead of having 60 mg of piroxicam and 240 mg of prilocaine, the patch may have 20 mg, 30 mg, 40 mg, 50 mg, 70 mg, 80 mg, 90 mg or 100 mg of piroxicam and 80 mg, 100 mg, 150 mg, 200 mg, 250 mg, 300 mg, 350 mg or 400 mg of prilocaine in any medically appropriate combination. The size of the patch can be 3 cm×5 cm, 5 cm×7 cm, 7 cm×10 cm, 10 cm×14 cm, etc.

Without being bound to the following theory, the active pharmaceutical ingredient (e.g., the piroxicam) is generally only poorly soluble in a suitable solvent therefor (e.g., water) at saturation before nanosizing. With nanosizing, the solubility increases significantly as the surface area of the active ingredient particles will have been increased significantly, and, thus, in certain respects the resultant preparation can be considered “super-saturated” relative to what it was before the nanosizing technique took place. In use as a pharmaceutical preparation for topical administration (e.g., as a transdermal patch, a gel or a cream), it is believed that there is a dynamic equilibrium between the suspended particles of the active pharmaceutical ingredient and the solubilized active pharmaceutical ingredient, such that, as the solubilized active pharmaceutical ingredient is transported through the skin, it is replenished in the solution by the suspended particles of the active pharmaceutical ingredient. It is believed, therefore, that the pharmaceutical preparation of the present invention serves as a self-replenishing depot for as long as the active pharmaceutical ingredient remains in suspension and for some time thereafter until all or substantially all of the active pharmaceutical ingredient is depleted. The same is true if a number of pharmaceutical active ingredients are in a topical preparation of the present invention and is applicable to active pharmaceutical ingredients (estrogens, nicotine, nitroglycerin, etc.) in many therapeutic areas (e.g., pain management, ophthalmology, etc.).

While various embodiments of the present invention have been described, it should be understood that various modifications and adaptations thereof will be apparent to one skilled in this art. Such modifications and adaptations are considered to be within the scope of the present invention, which is limited only by the scope of the following claims.

What is claimed is:

1. A pharmaceutical preparation for topical administration comprising an active pharmaceutical ingredient formulation in a pharmaceutically acceptable non-nanoparticle carrier therefor, wherein the active pharmaceutical ingredient formulation is a saturated solution of the active pharmaceutical ingredient in a solvent therefor in intimate combination and contact with a suspension of nanoparticles of the active pharmaceutical ingredient in the solvent.

2. The preparation of claim 1 wherein the active pharmaceutical ingredient is a non-steroidal anti-inflammatory drug (NSAID).

3. The preparation of claim 1 wherein the active pharmaceutical ingredient is a non-steroidal anti-inflammatory drug (NSAID) selected from the group consisting of aceclofenac, alminoprofen, apazone, aspirin, benoxaprofen, butibufen, carprofen, dexketoprofen, diclofenac, difenpiramide, diflunisal, droxicam, enbufen, etodolac, fenoprofen, flufenamic acid, flurbiprofen, ibuprofen, indomethacin, indoprofen, ketoprofen, ketorolac, lornoxicam, meclofenamic acid, mefenamic acid, meloxicam, nabumetone, naproxen, oxaprozin, phenylbutazone, piroxicam, pirprofen, pranoprofen, salicylic acid, sulindac, suprofen, tenoxicam, tiaprofenic acid, and tolmetin, and the pharmaceutically acceptable salts, esters or other derivatives thereof.

4. The preparation of claim 1 wherein the active pharmaceutical ingredient is selected from the group consisting of diclofenac, ketoprofen, ketorolac, and piroxicam.

5. The preparation of claim 1 wherein the active pharmaceutical ingredient is selected from the class of oxicam non-steroidal anti-inflammatory drugs (NSAIDs).

6. The preparation of claim 1 wherein the active pharmaceutical ingredient is piroxicam.

7. The preparation of claim 2 wherein the active pharmaceutical ingredient further includes a local anesthetic.

8. The preparation of claim 2 wherein the active pharmaceutical ingredient further includes a local anesthetic selected from the group consisting of articaine, benzocaine, bupivacaine, dibucaine, etidocaine, levobupivacaine, lidocaine, mepivacaine, piperocaine, prilocaine, ropivacaine, tetracaine, and trimecaine.

9. The preparation of claim 2 wherein the active pharmaceutical further includes a local anesthetic selected from the group consisting of bupivacaine, lidocaine, prilocaine and tetracaine.

10. The preparation of claim 2 wherein the active pharmaceutical further includes lidocaine.

11. The preparation of claim 4 wherein the active pharmaceutical further includes a local anesthetic selected from the group consisting of bupivacaine, lidocaine, prilocaine and tetracaine.

12. The preparation of claim 6 wherein the active pharmaceutical further includes lidocaine.

13. The preparation of claim 1 wherein the nanoparticles of the active pharmaceutical ingredient are less than 1000 nm in size.

14. The preparation of claim 1 wherein the nanoparticles of the active pharmaceutical ingredient are predominately in the range of 200-500 nm in size.

15. The preparation of claim 1 wherein the nanoparticles of the active pharmaceutical ingredient are predominately in the range of 1-200 nm in size.

16. The preparation of claim 1 wherein the nanoparticles of the active pharmaceutical ingredient are predominately in the range of 10-100 nm in size.

17. The preparation of claim 1 wherein the nanoparticles of the active pharmaceutical ingredient are in the range of 50-100 nm in size.

18. The preparation of claim 1 wherein the nanoparticles of the active pharmaceutical ingredient have a mean particle size of about 40-60 nm.

19. The preparation of claim 1 wherein the non-nanoparticle carrier is a cream, gel, lotion or transdermal patch.

20. The preparation of claim 1 wherein the non-nanoparticle carrier is a transdermal patch.

21. The preparation of claim 1 wherein the preparation is free of a penetration enhancer.

22. The preparation of claim 1 wherein the saturated solution of nanoparticles and the suspension of nanoparticles is prepared by dispersing solid particles of the active pharmaceutical ingredient in an aqueous solvent to create a suspension thereof with some particles having gone into solution and some particles remaining as solids carried by the solvent and subjecting the aqueous solution/suspension to a nanosizing technique.

23. The preparation of claim 22 wherein the nanosizing technique is irradiating the aqueous solution/suspension with a laser.

24. The preparation of claim 22 wherein the nanosizing technique is irradiating the aqueous solution/suspension with a pulse laser.

25. The preparation of claim 24 wherein the pulse laser is irradiated at an excitation light intensity of 1 to 1,000 mJ/cm².

26. The preparation of claim 22 wherein the nanosizing technique is irradiating the aqueous solution/suspension with a pulse laser having a pulse width ranging from several ten femtoseconds to several hundred nanoseconds.

27. The preparation of claim 22 wherein the saturated solution of nanoparticles and the suspension of nanoparticles is prepared by dispersing particles of the active pharmaceutical ingredient in an aqueous solvent to create a suspension thereof with some particles having gone into solution and some particles remaining as solids carried by the solvent and subjecting the aqueous solution/suspension to a nanosizing technique wherein, after the nanosizing technique is completed, the aqueous solution has more of the active pharmaceutical ingredient in solution compared to what was in solution before the nanosizing technique was undertaken and there are nanoparticles of the active pharmaceutical ingredient suspended in the aqueous solvent.

28. The preparation of claim 27 wherein the aqueous solvent is water.

29. The preparation of claim 27 wherein the nanoparticles of the active pharmaceutical ingredient suspended in the aqueous solvent after the nanosizing technique are less than 1000 nm in size.

30. The preparation of claim 27 wherein the nanoparticles of the active pharmaceutical ingredient suspended in the aqueous solvent after the nanosizing technique are predominately in the range of 10-100 nm in size.

31. The preparation of claim 27 wherein a majority of the nanoparticles of the active pharmaceutical ingredient suspended in the aqueous solvent after the nanosizing technique are predominately about 40-60 nm in size.

32. The preparation of claim 22 wherein the saturated solution of nanoparticles and the suspension of nanoparticles is prepared by dispersing solid particles of the active pharmaceutical ingredient in water to create a suspension thereof with some particles having gone into solution and some particles remaining as solids carried by the water and subjecting the water-based solution/suspension to a nanosizing technique wherein, after the nanosizing technique is completed, the water has more of the active pharmaceutical ingredient in solution compared to what was in solution before the nanosizing technique was undertaken and there are nanoparticles of the active pharmaceutical ingredient in the range of 10-100 nm suspended in the water.

33. The preparation of claim 32 wherein a majority of the nanoparticles of the active pharmaceutical ingredient suspended in the water after the nanosizing technique are predominately about 40-60 nm in size.

34. A method of treating acute and chronic pain and inflammation associated with rheumatoid arthritis, osteoarthritis, inflammatory arthropathies, gout and pseudogout, dysmenorrhea, metastatic bone pain, headache and migraine, postoperative pain, post-herpetic neuralgia, neuropathic pains, soft-tissue injuries, strains, sprains, contusions, tendonitis or bursitis of the shoulder, elbow, wrist or knee, Carpal tunnel syndrome, lateral epicondylosis, low back pains and injury comprising administering the preparation of claim 1.

35. A method of treating acute and chronic pain and inflammation associated with rheumatoid arthritis, osteoarthritis, inflammatory arthropathies, gout and pseudogout, dysmenorrhea, metastatic bone pain, headache and migraine, postoperative pain, post-herpetic neuralgia, neuropathic pains, soft-tissue injuries, strains, sprains, contusions, tendonitis or bursitis of the shoulder, elbow, wrist or knee, Carpal tunnel syndrome, lateral epicondylosis, low back pains and injury comprising administering the transdermal patch of claim 20.

36. A method of treating acute and chronic pain and inflammation associated with rheumatoid arthritis, osteoarthritis, inflammatory arthropathies, gout and pseudogout, dysmenorrhea, metastatic bone pain, headache and migraine, postoperative pain, post-herpetic neuralgia, neuropathic pains, soft-tissue injuries, strains, sprains, contusions, tendonitis or bursitis of the shoulder, elbow, wrist or knee, Carpal tunnel syndrome, lateral epicondylosis, low back pains and injury comprising administering the preparation of claim 22.

37. A method of treating acute and chronic pain and inflammation associated with rheumatoid arthritis, osteoarthritis, inflammatory arthropathies, gout and pseudogout, dysmenorrhea, metastatic bone pain, headache and migraine, postoperative pain, post-herpetic neuralgia, neuropathic pains, soft-tissue injuries, strains, sprains, contusions, tendonitis or bursitis of the shoulder, elbow, wrist or knee, Carpal tunnel syndrome, lateral epicondylosis, low back pains and injury comprising administering the preparation of claim 32.
